(19) United States
(12) Patent Application Publication
(10) Pub. No.: US 2026/0335119 A1
(43) Pub. Date: Oct. 29, 2026

(54) Apparatus For Permanently Extending A Penis

(71) Applicant: SWISS-TEC GLOBAL LIMITED, Vaduz (LI)

(72) Inventor: Julian Jochum, Pöcking (DE)

(21) Appl. No.: 19/104,067

(22) PCT Filed: Aug. 16, 2023
(86) PCT No.: PCT/EP2023/072584

§ 371 Date: Feb. 14, 2025

(30) Foreign Application Priority Data
Aug. 17, 2022 (DE) 10 2022 120 804.4

(51) Int. Cl. A61F 5/41

(52) U.S. Cl. CPC A61F 5/41; A61F 2005/411

(57) ABSTRACT

The invention relates to an apparatus (1) for permanently extending a penis, which apparatus comprises a support ring (2) and at least two extension rods (3) which are articulatedly fastened to the support ring (2) and are axially resiliently mounted by means of a spring (42) and are longitudinally adjustable, wherein the extension rods (3) comprise: a joint sleeve (10), which is articulatedly connected at one end to the support ring (2); a spring sleeve (34), in which the spring (42) is arranged and which is connected at one end to a coupling element (4) suitable for connecting a fastening device (43) for a penis to the extension rods (3) and for exerting a tensile force on the penis; and at least one rod-shaped telescopic adjustment unit arranged between the spring sleeve (34) and the joint sleeve (10). The problem addressed by the invention is solved in that: the joint sleeve (10) comprises an internal bore (19), in the wall of which an internal thread (19) is formed; a first (20) and a second (25) telescopic adjustment unit are arranged between the spring sleeve (34) and the joint sleeve (10); the first adjustment unit provides a threaded sleeve (20) which has an external thread (21), that is screwed into the internal thread (19) of the joint sleeve (10), and an internal bore (22), in the wall of which an internal thread (23) is formed; the second adjustment unit provides a threaded spindle (25) which, with respect to the threaded sleeve (20), is fitted with an external thread (27) that is screwed into the internal thread (23) of the threaded sleeve (20) and, with respect to the spring sleeve (34), is fitted with a pin (28) projecting into the spring sleeve (34); and the spring (42) is arranged in the spring sleeve (34) between the pin (28) and the coupling element (4).

### DESCRIPTION

The invention relates to an apparatus for permanently extending a penis, which apparatus comprises a support ring and at least two extension rods which are articulatedly fastened to the support ring and are axially resiliently mounted by means of a spring and are longitudinally adjustable, wherein the extension rods comprise a joint sleeve which is articulatedly connected at one end to the support ring, a spring sleeve in which the spring is arranged and which is connected at one end to a coupling element which is suitable for connecting a fastening device for a penis to the extension rods and for exerting a tensile force on the penis, and at least one rod-shaped telescopic adjustment unit arranged between the spring sleeve and the joint sleeve.

Apparatuses of the type in question here, which are also referred to as penis extension apparatuses, are known in a variety of embodiments. For example, EP 3463218 B1 describes a fastening device of such a penis extension apparatus with a tension meter. Numerous relevant apparatuses are also described and acknowledged in this publication. Reference is hereby made to these explanations.

An apparatus for exerting traction on a penis is known from US 2004/0215055A1 , which has a ring-shaped base element and two connecting elements articulatedly fastened to the base element. These connecting elements of the known traction apparatus are provided with a threaded rod which can be screwed into a piston. The end of the piston comes into contact with a spring mounted in a spring sleeve, by means of which a tensile force can be exerted in the longitudinal direction of the extension rod.

Several rod-shaped extension elements, which have a threaded pin at one end and a threaded hole at the other end, are used to adjust the length of the extension rods in this known tensioning apparatus. To connect these extension elements to each other, one extension element is screwed with its threaded pin into the threaded hole of the adjacent extension element. A third extension element can be added to a combination of two extension elements connected in this way in the manner described.

The total length of the final threaded rod is determined by the number and length of the individual extension elements connected together in this way. The unit of several interconnected extension elements is connected at one end to the spring sleeve and at the other end to a fastening device for the penis.

The threaded pins of the extension elements are fully screwed into the corresponding threaded holes to ensure the required stability of the unit, which consists of several extension elements. There is no provision for adjusting the length by turning the threaded hole in relation to the threaded pin. Lengthening or shortening of the extension rods can therefore only be achieved by adding or removing extension elements and thus, for example, not while the user is wearing this known extension apparatus.

DE 202 03 927 U1 describes a special embodiment of a generic penis extension apparatus which corresponds to the preamble of claim 1.

Based on the latter prior art, it is the object of the present invention to provide an apparatus of the type in question here, which can be easily and flexibly adapted to the length requirements.

This object is solved by an apparatus which is characterized in that the joint sleeve comprises an internal bore in the wall of which an internal thread is formed,
a first and a second telescopic adjustment unit are arranged between the spring sleeve and the joint sleeve,
the first adjustment unit provides a threaded sleeve which has an external thread which is screwed into the internal thread of the joint sleeve and an internal bore in the wall of which an internal thread is formed,
the second adjustment unit provides a threaded spindle, which is provided towards the threaded sleeve with an external thread, which is screwed into the internal thread of the threaded sleeve, and towards the spring sleeve with a plug pin projecting into the spring sleeve, and
the spring is arranged in the spring sleeve between the plug pin and the coupling element.

Preferably, a radially outer circumferential stop flange is formed between the external thread and the plug pin of the threaded spindle.

Furthermore, the threaded sleeve also preferably has a radially outer circumferential stop flange at its distal end.

The length of the threaded spindle, threaded sleeve and joint sleeve are suitably selected and matched to each other so that the desired and/or required adjustment range is covered.

The internal thread of the joint sleeve is formed in the wall of the internal bore of the joint sleeve. The internal bore is open distally and closed proximally, but can also be open.

Preferably, the axial length of the internal bore of the joint sleeve makes up at least 80% of the axial length of the joint sleeve.

The internal thread can extend over the entire axial length of the internal bore or only over a section or region thereof.

The threaded sleeve also has an internal bore, which is open distally and can be closed or open proximally. The internal thread of the threaded sleeve is formed in the wall of the internal bore and can extend over the entire axial length of the internal bore or only over a section or region of the internal bore.

Preferably, the axial length of the internal bore of the threaded sleeve is at least 80% of the axial length of the threaded sleeve.

The threaded sleeve also has an external thread which is formed on the outer surface of the threaded sleeve and which can extend over the entire axial length of the threaded sleeve or only a region or section thereof.

Preferably, the axial length available for forming the external thread of the threaded sleeve is at least 80% of the total axial length of the threaded sleeve.

Preferably, the axial length of the outer surface of the threaded sleeve, on which the external thread can be formed, is at least 80% of the axial length of the internal bore of the joint sleeve.

Furthermore, the axial length available for forming the external thread of the threaded spindle is preferably at least 80% of the axial length of the internal bore of the threaded sleeve.

The region of the threaded spindle, on which the external thread of the threaded spindle can be formed, extends from the plug pin to the proximal end or over a section or region thereof.

The preferred features described above are intended to ensure that the two adjustment units, namely the threaded sleeve and threaded spindle, and the joint sleeve can preferably be screwed into each other as far as possible and also unscrewed again, so that in the screwed-in state the threaded sleeve is at least 80% and even completely screwed into the joint sleeve and the threaded spindle is at least 80% and even completely screwed into the threaded sleeve.

If preferred stop flanges are present, these are of course not screwed in anywhere.

When unscrewed, the length of the unit consisting of the joint sleeve and the two adjustment units is as long as possible. This results in a very large adjustment range overall.

The term “unscrewed state” refers to a situation in which the elements that are screwed together are still connected and therefore not separated from each other, but are as far apart as possible in the longitudinal direction.

The spring sleeve is preferably closed towards the coupling element by a latchable cover and the spring is arranged between the cover and the plug pin.

An apparatus is particularly preferred which is characterized in that the extension rods are fastened to the support ring in such a way that the support ring has two parallel jaws per extension rod, each of which has an inwardly open recess, that the joint sleeves have a joint piece which has an annular disk-shaped central part on which a hemispherical bulge is formed on both sides, the thickness of the annular disk-shaped central part is less than the distance between the jaws and the bulges engage in the opposite recess.

The coupling element of the apparatus according to the invention is preferably connected to a fastening device for a penis. This fastening device is of a known nature and is described, for example, in EP 3 463 218B1 .

According to an alternative embodiment, the coupling element as such can be designed as a fastening device for a penis. Such a fastening device is also known, for example from DE 202 03 927U1 .

The term “spring” used here stands for an elastic element that develops a restoring force to the initial position when compressed. The spring of the apparatus according to the invention is preferably a compression spring or a helical spring made in particular of a metal, a rod or a tube made of an elastic material.

A foam material and preferably silicone can serve as an elastic material.

The other elements of the apparatus according to the invention are, in particular, injection-molded parts made of a known plastic material suitable for this purpose.

This plastic material can be a transparent material. This applies in particular to the joint sleeve and the threaded sleeve, as this makes it possible to visually determine how far the threaded sleeve is screwed into the joint sleeve or the threaded spindle is screwed into the threaded sleeve.

The invention is described in more detail below with reference to the exemplary drawings, which are not true to scale, wherein the figures show as follows:

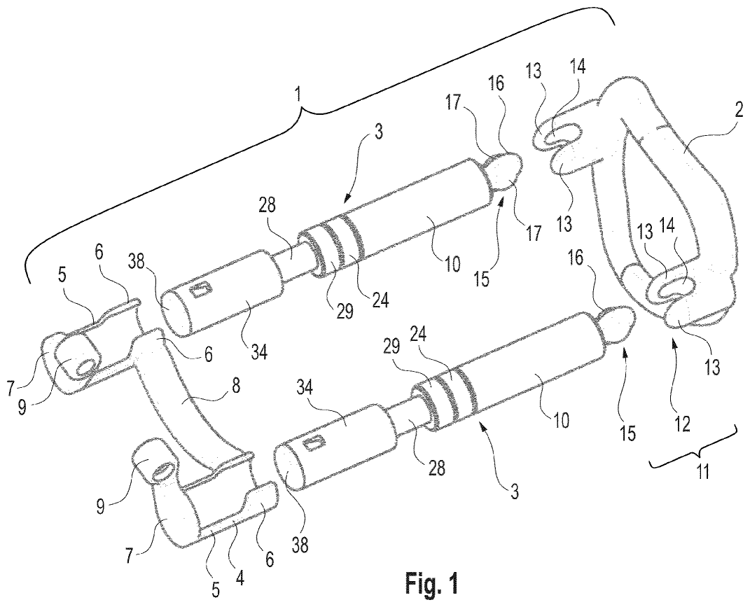

FIG. 1 shows a top view of an apparatus according to the invention, partly in exploded view,

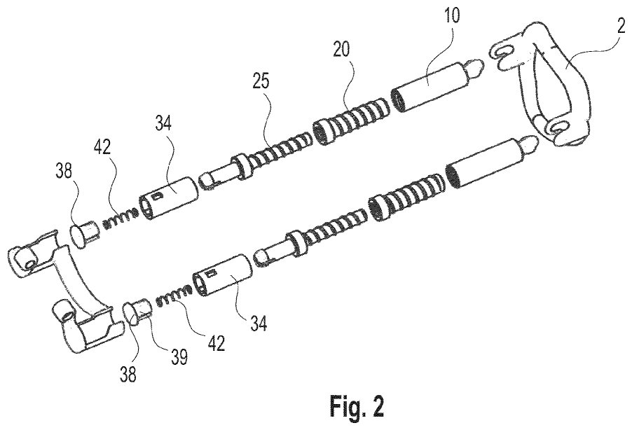

FIG. 2 shows a view corresponding to FIG. 1, but as a complete exploded view,

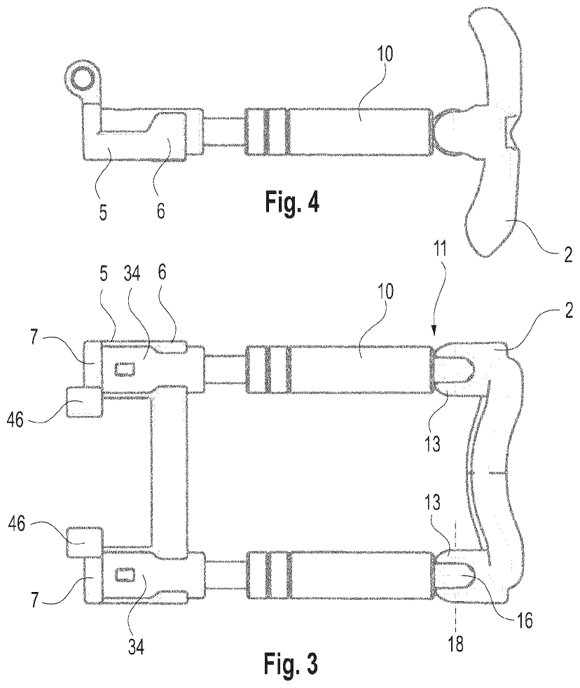

FIG. 3 shows a top view of the apparatus shown in FIG. 1 in the assembled state, in which the threaded sleeve and the two adjustment units are screwed into each other,

FIG. 4 shows a side view of the apparatus shown in FIG. 3,

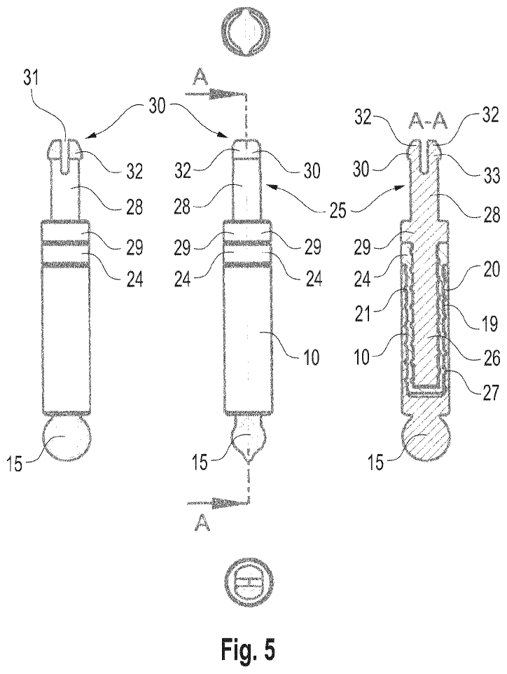

FIG. 5 shows two side views of a joint sleeve into which a first adjustment unit is completely screwed in, wherein a second adjustment unit is completely screwed into the first adjustment unit, and a longitudinal section along A-A,

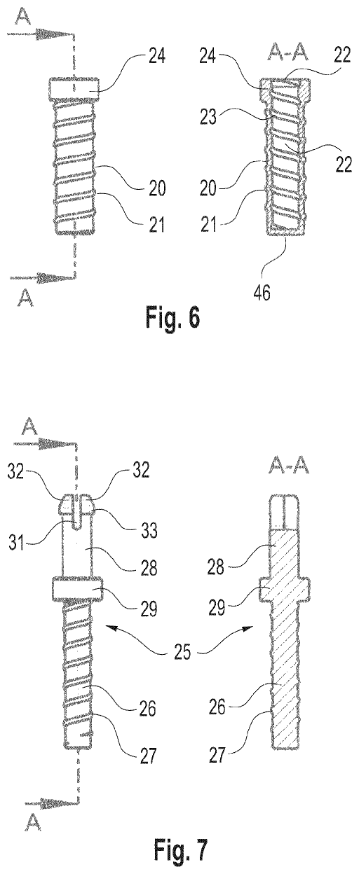

FIG. 6 shows a side view of the first adjustment unit in the form of a threaded sleeve and a longitudinal section along A-A,

FIG. 7 shows a side view of a second adjustment unit in the form of a threaded spindle and a longitudinal section along A-A,

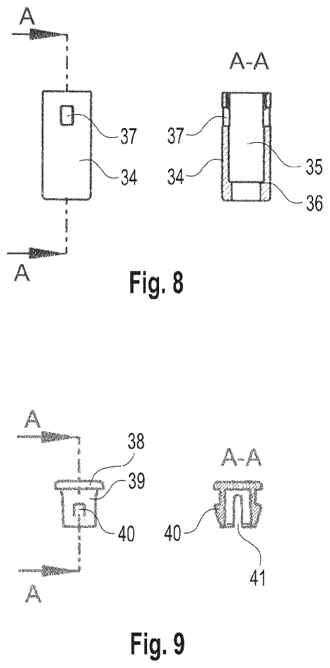

FIG. 8 shows a side view of a spring sleeve and a longitudinal section along A-A,

FIG. 9 shows a side view of a cover for the spring sleeve and a longitudinal section along A-A and

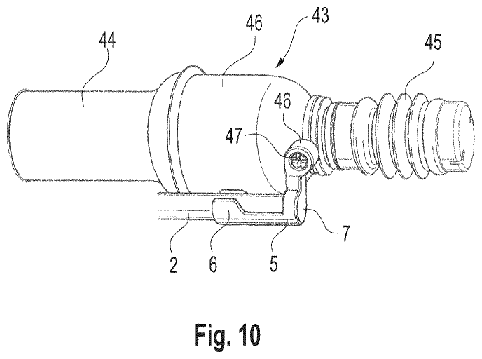

FIG. 10 shows a perspective view of a fastening device for connecting a penis to a penis extension apparatus, wherein this fastening device is known, inter alia, from EP 3 463 218B1 .

The apparatus 1 according to the invention, shown in FIG. 1 in an oblique view from above, has three components, namely a support ring 2, two length-adjustable extension rods 3 and a coupling element 4. These three components are shown separately and therefore not connected to each other,

The support ring 2 is used to support the apparatus 1 on the user's body and represents the proximal part of the apparatus 1. Such a support ring 2 is known from the prior art described above.

The distal end of the extension rods 3 opposite the support ring 2 is arranged in a cylindrical receiving part 5 of the coupling element 4, which is closed distally by a cover 7 and open laterally in the longitudinal direction. The receiving part 5 has resilient cheeks 6, which are described in more detail below. A connecting element 8, which is attached at both ends to the associated cheek 6, is used to connect the two receiving parts 5. A bearing bush 9 is attached to the cover 7. The elements of the coupling element 4 described above are injection molded in one piece.

Two multi-element extension rods 3 are pivotably attached to the support ring 2.

At the proximal end, the extension rod 3 has a joint sleeve 10, which is pivoted at its proximal end to the support ring 2 by means of a joint 11 and is articulated with lateral play.

The joint 11 has a joint holder 12, which is attached to the support ring 2. Each joint holder 12 has two jaws 13 aligned parallel to each other, each of which has a recess 14 that is open inwards and therefore open towards the opposite jaw 13. Support ring 2 together with jaws 13 are injection molded in one piece.

The joint 11 also includes a joint piece 15 on the joint sleeve 10, which has an annular disk-shaped central part 16, on both sides of which a hemispherical bulge 17 is formed.

As can be seen in particular from FIG. 3, when the joint 11 is assembled, the central part 16 comes to lie between the two opposing jaws 13 of the joint holder 12, wherein the hemispherical bulges 17 are arranged in the opposing recess 14 of the jaw 13. The thickness of the central part 16 is less than the distance between the two jaws 13, which means that the joint sleeve 10 and thus the extension rods 2 can not only rotate about the axis 18 extending through the two recesses 14, but can also pivot in the direction of this axis 18.

As can be seen in particular from FIGS. 2 and 5, the joint sleeve 10 has a threaded internal bore 19 extending in the longitudinal direction, which is open towards its distal end.

A first telescopic adjustment unit in the form of a hollow cylindrical threaded sleeve 20, shown in more detail in FIG. 6, is screwed into the internal bore 19 of the joint sleeve 10. The threaded sleeve 20 is equipped with an external thread 21 on its outer jacket surface, which is dimensioned and shaped in such a way that it can be screwed into the thread of the internal bore 19 of the joint sleeve 10. The internal bore 19 is closed proximally by a wall 46 and represents a type of blind bore.

The threaded sleeve 20 also has a cylindrical internal bore 22 extending in the longitudinal direction and provided with a thread 23, as well as a radially circumferential stop flange 24 extending radially outwards at its distal end.

A second telescopic adjustment unit in the form of a threaded spindle 25 (see in particular FIG. 7) is connected distally to the threaded sleeve 20. This threaded spindle 25 has two sections, namely a proximal section in the form of a cylindrical pin 26 with an external thread 27 and a distal section in the form of a cylindrical plug pin 28. A radially circumferential stop flange 29 extending radially outwards is arranged between these two sections.

The external thread 27 of the threaded spindle 25 is dimensioned and shaped in such a way that the threaded spindle 25 can be screwed into the threaded sleeve 20 until the stop flange 29 of the threaded spindle 25 comes into contact with the stop flange 24 of the threaded sleeve 20. The two stop flanges 24 and 29 have the same outer diameter and the same axial thickness. This outer diameter corresponds to the outer diameter of the joint sleeve 10.

At the distal end of the threaded spindle 25, a latching tip 30 is arranged, which has a notch 31 extending in the longitudinal direction, whereby two opposing latching tongues 32 are formed. A radially outwardly pointing and radially circumferential latching lug 33 is formed at the transition from the latching tongue 32 to the plug pin 28.

The threaded spindle 25 is distally adjoined by an outer cylindrical spring sleeve 34 (see in particular FIG. 8), which has a continuous cylindrical internal bore 35 arranged in the longitudinal direction. The diameter of this internal bore 35 widens in the distal direction in the form of a shoulder 36. When the latching tip 30 is inserted into the proximal end of the spring sleeve 34, the two latching tongues 32 are pressed together until they reach the area with the larger diameter of the internal bore 35 and move away from each other again. The latching lugs 33 resting on the shoulder 30 prevent the latching tip 30 and thus the threaded spindle 25 from being pulled out.

The spring sleeve 34 has two diametrically opposed bores 37 with an approximately rectangular shape in plan view, which extend through the wall of the spring sleeve 34. The distal end of the spring sleeve 34 is closed by a cover 38, shown in more detail in FIG. 9, which has a hollow cylindrical extension 39 which is dimensioned and shaped in such a way that it can be inserted into the distal end of the spring sleeve 34. The free end of this extension 39 is provided with an incision 41 and has two latching lugs 40, which engage in the associated bore 37 in the spring sleeve 34.

A compression spring 42 is inserted into the spring sleeve 34, which is located between the cover 38 and the latching tip 30.

In one embodiment, the coupling element 4 serves to connect a fastening device 43 known per se to the coupling element 4. In FIG. 10, a fastening device 43 of this type is known inter alla from EP 3 463 218B1, which has a flexible sleeve 44 and a hollow body 46 for holding a penis. After inserting the penis into the hollow body 46, the sleeve 44 is attached to the penis, for example by rolling the sleeve 44, which has previously been rolled onto the hollow body 46, onto the penis. The vacuum pump 45 can generate a vacuum so that a stable connection is created between the penis and the fastening device 43. All this is known.

The fastening device 43 is connected to the coupling element 4 of the apparatus 1 according to the invention via the hollow cylindrical receiving part 5, which is equipped with a cover 7. A bearing bush 9 (see also FIG. 1) is attached or formed on this cover 7, in which a bearing journal 47 is rotatably mounted, which is connected to the fastening device 43.

The apparatus 1 according to the invention can be attached as follows, Each extension rod 3 is mounted in the corresponding joint 11 by inserting the joint piece 15 between the jaws 13 and expanding it. The threaded sleeve 20 is also twisted or screwed into the joint sleeve 10. In addition, the threaded spindle 25 with its cylindrical pin 26 is twisted or screwed into the threaded sleeve 20. The plug pin 28 of the threaded spindle 25 is inserted into the spring sleeve 34 until the latching tip 30 engages inside the spring sleeve 34. The compression spring 42 is inserted into the spring sleeve 34. The internal bore 35 of the spring sleeve 34 is then closed by the cover 38, which engages in the bores 37 with its latching lugs 40. The cover 38 is spaced from the latching lug 33. This space contains the compression spring 48, which is compressed when the plug pin 28 is pushed into the spring sleeve 34.

The spring sleeve 34 is pushed into the associated hollow cylindrical receiving part 5 or clipped into place while expanding the resilient cheeks 6. In the assembled state, the cover 38 lies against the inside of the axially opposite cover 7 of the receiving part 25. This assembled state of the apparatus 1 according to the invention is shown in FIGS. 3 and 4, among others. These processes are carried out for each extension rod 3.

The bearing journals 47 attached to the connecting apparatus 43 are inserted or pushed into the bearing bushes 9 attached to the covers 7. This state is shown in FIG. 10.

It is evidently also possible to first connect the fastening device 43 to the coupling element 4 in the manner described above and then insert the spring sleeve 34 into the receiving parts 5.

The penis can be inserted into the flexible sleeve 44, which is a type of condom, and the hollow body 48 before or after assembly of the apparatus according to the invention.

Incidentally, it is also possible to use the fastening means known from DE 202 03 927 U1 instead of the fastening device 43 described above in order to connect the penis to the apparatus 1. In this case, the coupling element 4 is a fastening means according to DE 202 03 927U1 .

In order to exert a tensile force on the penis, either the joint sleeve 10 or the threaded spindle 25 or both the joint sleeve 10 and the threaded spindle 25 are unscrewed from their furthest screwed-in position (see FIGS. 3 and 4). This extends the axial length of the threaded sleeve 10 plus the threaded spindle 25 and consequently the extension rods 3. This exerts pressure on the compression spring 42 in the direction of the coupling element 4, whereby a tensile force is exerted on the penis by means of the fastening device 43.

This tensile force can be finely adjusted due to the design of the extension rods 3. In addition, due to two telescopic adjustment units in the form of a threaded sleeve 20 and a threaded spindle 25 in combination with a joint sleeve 10, the apparatus according to the invention can be used in a wide range of lengths in order to meet the various requirements. This means that different penis sizes can be accommodated with one apparatus.

The axial length of the threaded sleeves 20 and the threaded spindle 25 can be selected as required. This means that short and also longer threaded sleeves 20 and short and also longer threaded spindles 25 can be used for an apparatus 1,

### LIST OF REFERENCE SIGNS

1 Apparatus
2 Support ring
3 Length-adjustable extension rod
4 Coupling element
5 Hollow cylindrical receiving part
6 Resilient cheek
7 Cover
8 Connecting element
9 Bearing bush
10 Joint sleeve
11 Joint
12 Joint holder
13 Cheek
14 Recess
15 Joint piece
16 Central part
17 Hemispherical bulge
18 Axis
19 Internal bore with thread of the joint sleeve 10
20 Hollow cylindrical threaded sleeve
21 External thread of the threaded sleeve 20
22 Internal bore of the threaded sleeve 20
23 Thread of internal bore 22
24 Stop flange
25 Threaded spindle
26 Cylindrical pin of threaded spindle 25
27 External thread of threaded spindle 25
28 Cylindrical plug pin of threaded spindle 25
29 Stop flange of threaded spindle 25
30 Latching tip
31 Incision in latching tip 30
32 Latching tongue
34 Spring sleeve
35 Internal bore of spring sleeve 34
36 Shoulder in internal bore 35
37 Bore
38 Cover
39 Hollow cylindrical extension of cover 38
40 Latching lug
41 Incision in hollow cylindrical extension 39
42 Compression spring
43 Fastening device
44 Flexible sleeve
45 Vacuum pump
46 Hollow body
47 Bearing journal

1. An apparatus for permanently extending a penis, which comprises a support ring and at least two extension rods which are articulatedly fastened to the support ring and are axially resiliently mounted by means of a spring and are longitudinally adjustable, wherein the extension rods comprise a joint sleeve which is articulatedly connected at one end to the support ring, a spring sleeve in which the spring is arranged and which is connected at one end to a coupling element which is suitable for connecting a fastening device for a penis to the extension rods and for exerting a tensile force on the penis, and at least one rod-shaped telescopic adjustment unit arranged between the spring sleeve and the joint sleeve, wherein:
the joint sleeve comprises an internal bore in the wall of which an internal thread is formed,
a first and a second telescopic adjustment unit are arranged between the spring sleeve and the joint sleeve,
the first adjustment unit provides a threaded sleeve which has an external thread which is screwed into the internal thread of the joint sleeve and an internal bore in the wall of which an internal thread is formed,
the second adjustment unit provides a threaded spindle, which is provided towards the threaded sleeve with an external thread, which is screwed into the internal thread of the threaded sleeve, and towards the spring sleeve with a plug pin projecting into the spring sleeve, and
the spring is arranged in the spring sleeve between the plug pin and the coupling element.

2. The apparatus according to claim 1, wherein a radially outer circumferential stop flange is formed between the external thread and the plug pin of the threaded spindle.

3. The apparatus according to claim 1, wherein
the axial length of the internal bore of the joint sleeve makes up at least

4. The apparatus according to claim 1, wherein the spring sleeve is closed towards the coupling element by a latchable cover and the spring is arranged between the cover and the plug pin.

5. The apparatus according to claim 1, wherein, the extension rods are fastened to the support ring in such a way that the support ring has two parallel jaws per extension rod, each of which has an inwardly open recess, and wherein the joint sleeves have a joint piece, which has an annular disk-shaped central part, on which a hemispherical bulge is formed on both sides, the thickness of the annular disk-shaped central part being less than the distance between the jaws and the bulges engage in the opposite recess.

6. The apparatus according to claim 1, wherein, the coupling element is connected to a fastening device for a penis.

7. The apparatus according to claim 1, wherein the coupling element is a fastening device for a penis.

8. The apparatus according to claim 1, wherein the spring is a compression spring, a rod made of an elastic material or a tube made of an elastic material.

9. The apparatus according to claim 8, wherein the elastic material is silicone.

10. The apparatus according to claim 1, wherein the axial length of the internal bore of the joint sleeve makes up at least 80% of the axial length of the joint sleeve.

11. The apparatus according to claim 1, wherein the axial length of the internal bore of the threaded sleeve makes up at least 80% of an axial length of the threaded sleeve.

12. The apparatus according to claim 1, wherein the axial length available for forming the external thread of the threaded sleeve is at least 80% of a total axial length of the threaded sleeve.

13. The apparatus according to claim 1, wherein the axial length of the outer surface of the threaded sleeve, which is available for the formation of the external thread, makes up at least 80% of a total axial length of the internal bore of the threaded sleeve.

14. The apparatus according to claim 1, wherein an axial length available for forming the external thread of the threaded spindle is at least 80% of the axial length of an internal bore of the threaded sleeve.

15. An apparatus for permanently extending a penis, the apparatus comprising:
a support ring;
a spring;
at least two extension rods which are articulatedly fastened to the support ring, axially resiliently mounted by means of the spring and longitudinally adjustable, wherein the extension rods comprise:
a joint sleeve which is articulatedly connected at one end to the support ring, the joint sleeve comprising an internal bore with a wall in which an internal thread is formed,
a spring sleeve in which the spring is arranged and which is connected at one end to a coupling element which is suitable for connecting a fastening device for a penis to the extension rods and for exerting a tensile force on the penis, and
a first and a second telescopic adjustment unit arranged between the spring sleeve and the joint sleeve, wherein:
the first adjustment unit provides a threaded sleeve which has an external thread which is screwed into the internal thread of the joint sleeve and an internal bore with a wall in which an internal thread is formed,
the second adjustment unit provides a threaded spindle, which is provided towards the threaded sleeve with an external thread, which is screwed into the internal thread of the threaded sleeve.

16. The apparatus of claim 15, wherein the threaded sleeve has a radially outer circumferential stop flange at its distal end.

17. The apparatus according to claim 15, wherein an axial length of the internal bore of the joint sleeve makes up at least 80% of an axial length of the joint sleeve.

18. The apparatus according to claim 15, wherein an axial length of the internal bore of the threaded sleeve makes up at least 80% of an axial length of the threaded sleeve.

19. The apparatus according to claim 15, wherein an axial length available for forming the external thread of the threaded sleeve is at least 80% of a total axial length of the threaded sleeve.

20. The apparatus according to claim 15, wherein the extension rods are fastened to the support ring in such a way that the support ring has two parallel jaws per extension rod, each of which has an inwardly open recess, and wherein the joint sleeves have a joint piece, which has an annular disk-shaped central part, on which a hemispherical bulge is formed on both sides, the thickness of the annular disk-shaped central part being less than the distance between the jaws and the bulges engage in the opposite recess.
